(19) United States
(12) Patent Application Publication
(10) Pub. No.: US 2005/0273107 A1
(43) Pub. Date: Dec. 8, 2005

(54) Cannulated drill-pin implant with related systems and methods

(76) Inventor: Peter M. Stevens, Salt Lake City, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/198,637

(22) Filed: Aug. 5, 2005

Related U.S. Application Data
(63) Continuation-in-part of application No. PCT/IB04/00429, filed on Feb. 20, 2004.
(60) Provisional application No. 60/449,327, filed on Feb. 22, 2003.

(51) Int. Cl. A61B 17/86

(52) U.S. Cl. 606/73; 606/80

(57) ABSTRACT

A drill-pin implant includes a cannulated body having an interior surface and an exterior surface each extend between a first end and an opposing second end. The interior surface bounds a guide aperature through the body. The first end of the body is configured to penetrate into bone by cutting away the bone as the body is rotated. One or more external threads redially outwardly project from the exterior surface of the body. The external threads are configured to engage bone so as to secure the body to the bone.

### CROSS-REFERENCE TO RELATED APPLICATIONS

The present application is a continuation-in-part of International Application No. PCT/IB2004/000429, Filed Feb. 20, 2004 which claims priority to U.S. Provisional Application No. 60/449,327, filed Feb. 22, 2003.

### BACKGROUND OF THE INVENTION

1. The Field of the Invention

This invention relates to mechanical fasteners for surgical use and, more particularly, to half pin fasteners for bone fixators.

2. The Relevant Technology

Standard half pins are employed for the fixation of long bones that have been fractured or surgically divided. Half pins are typically non-cannulated and are threaded to engage both sides of the bone and prevent slippage. Typically, a hole must first be drilled in the bone. Then, the drill bit is removed and the half pin inserted.

Problems associated with this method of application include dull drill bits, heat generation (burned bone), eccentric pin placement and multiple steps involved in the insertion of each pin. As a result, surgical time and radiographic imaging exposure are increased and secure fixator placement may be compromised. Suboptimal pin placement may result in pin loosening, pin breakage, stress fracture of the bone, or bone fragmentation (i.e., ring sequestrum), therefore ultimately requiring secondary surgery.

External frames (fixators) are ubiquitous in the orthopedic armamentarium for managing long bone fractures and/or for limb lengthening or deformity correction. The frames are attached to long bone(s) via multiple half pins that are inserted percutaneously (i.e., blindly) into bone segments. Central and concentric pin placement requires skill and intuition and is usually documented by fluoroscopy. Frames are typically worn for weeks, months or in some instances, more than a year. The outcome of this treatment technique is predicated upon secure and accurate frame attachment and is reliant upon optimization of the pin-bone interface. Eccentric pin placement causes pin related problems including drainage and infection. Consequently, bending or breakage of pins is relatively common, thereby complicating this method of treatment.

A variety of orthopedic half pins are available on the market. While some are tapered or self-tapping, all require pre-drilling of the bone prior to pin insertion. A common pitfall of drilling bone, exacerbated by dull drill bits, is the potential to drill eccentric holes, compromising the integrity of the bone and the strength of fixation. This is often unrecognized until later when the bone/pin interface fails due to, for instance, dead bone (heat necrosis), secondary infection, pin fatigue, or stress fracture of the bone. These situations typically mandate secondary, unplanned surgical intervention to revise pin placement and alter the frame construct. Such unanticipated steps in fracture management or deformity correction may comprise the outcome of the surgery.

What is needed is a fastener that overcomes these limitations in a product which minimizes surgical time and radiographic image exposure, while providing a reliable, accurate and secure placement of the half pin fastener. Such a fastener should also lend itself to use with most makes and models of fixators.

### SUMMARY OF THE INVENTION

In one embodiment, a drill-pin implant, which meets the needs identified above, has an elongated, cylindrical body extending along a central axis. The cylindrical body has opposing end portions, namely an operative end portion or tip and a tool engaging portion or application end. The operative end portion is formed so as to be suitable for penetrating bone. The tool engaging end portion is formed so as to be suitable for engagement with a hand or power drill. The body is cannulated through its entire length to permit drill-pin insertion over a guide pin. The body further has an external threaded portion suitable for engaging with the bone, in order to prevent the implant from slipping. The thin, unobtrusive guide pin is first inserted into the bone, documenting precise and concentric placement with radiographic imaging. The drill-pin implant is inserted over the guide pin and advanced until the drill tip penetrates the far side of the bone and the external threads engage the near side. The guide pin is then removed. A bone plate or fixator may then be affixed to the implant, as appropriate, and the incision closed.

The inventive drill-pin implant and related system ensures efficient and accurate placement of bone implants.

In an advantage of the invention, the drill is “disposable” (in that it is for single use), it is always sharp and of known true diameter, thus reducing the likelihood of intraoperative or postoperative complications such as burned bone or stress fractures.

In a further advantage the drill-pin serves as the implant, thus saving the cost of stocking and sharpening multiple drill bits and carrying an expanded inventory of pins.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a side view of a drill bit of the prior art in a bone;

FIGS. 2A-2C are schematic views showing complications which arise due to eccentric pin placement;

FIG. 3A is a side view of one embodiment of a drill-pin implant of the present invention;

FIG. 3B is a cross sectional view of the drill-pin implant taken along line 3B-3B of FIG. 3A;

FIG. 3C is a perspective view of the end of the drill-pin implant shown in FIG. 3A;

FIG. 4 is a side view of one embodiment of guide pin that can be used with the drill-pin implant of FIG. 3A;

FIG. 5 is a flow chart of one method of use of the invention;

FIGS. 6A-6C are schematic views showing a first step of a method of use of one embodiment of the present invention;

FIGS. 7A-7C are schematic views showing a second step of a method of use;

FIGS. 8A-8C are schematic views showing a third step of a method of use;

FIG. 9A is an elevated side view of an alternative embodiment of a drill-pin implant;

FIG. 9B is an enlarged side view of the lead end of the drill-pin implant shown in FIG. 9A;

FIG. 9C is an elevated front end view of the drill-pin implant shown in FIG. 9A;

FIG. 9D is an enlarged cross sectional side view of the external threads of the drill-ant pin implant shown in FIG. 9A; and

FIG. 10 is an elevated side view of a tubular drill sleeve having the drill-pin implant of FIG. 9A disposed therein.

### DETAILED DESCRIPTION OF THE PREFERRED EMBODIMENTS

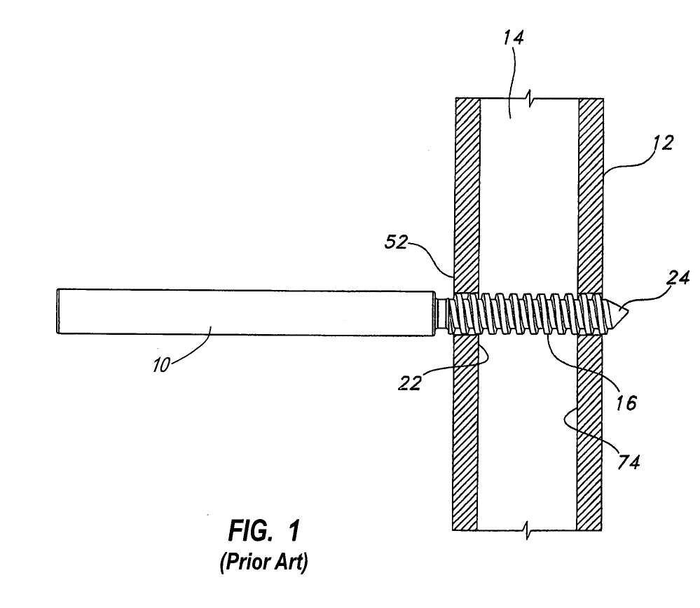

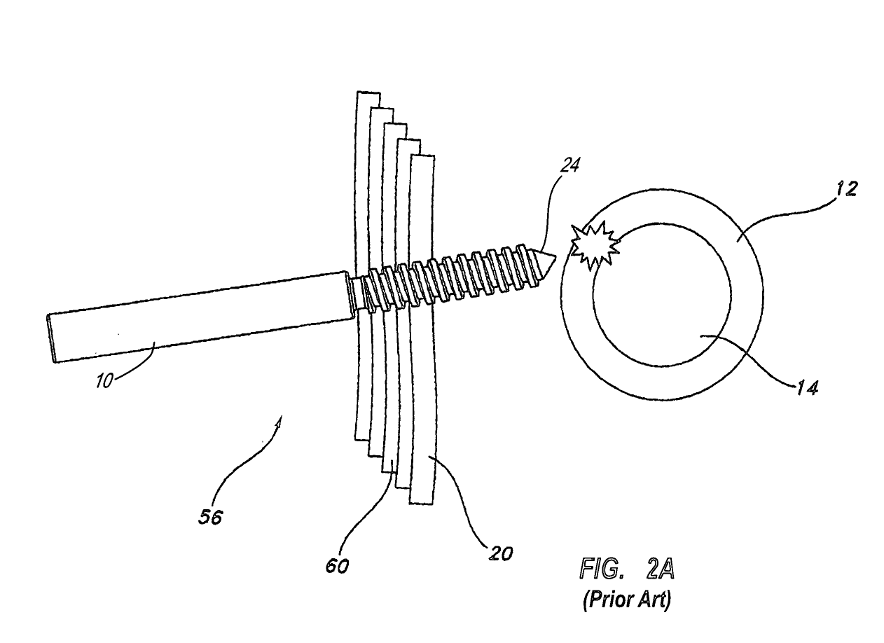

Now referring to FIG. 1, a standard drill bit 10 of the prior art is shown having penetrated a bone 12 with a cortex 14. The prior art drill bit 10 is long, cylindrical and has extended cutting flutes 16 which often traumatize soft tissue 20 (shown in FIG. 2A).

Now referring to FIGS. 2A-2C, the drill bit 10, which is non-cannulated and may or may not be sharp, may “walk” or wander before penetrating the near cortex 22 of the bone 12. As a result, heat generation may dull the tip 24 of the drill bit 10 and burn the bone 12 with the result that accurate, concentric pin placement may be impossible. This problem is accentuated with each successive use of the drill bit 10 during the same or subsequent patients.

Referring in particular to FIG. 2B, eccentric placement of the half pin implant (not shown, but in the same position as drill bit 10) may result in premature pin loosening and/or stress fracture of the bone 12 under repetitive loading conditions. Since these pins are in place for weeks or months (or longer) cumulative stresses may result in failure of the pin or bone 12 and necessitate secondary and unplanned surgery.

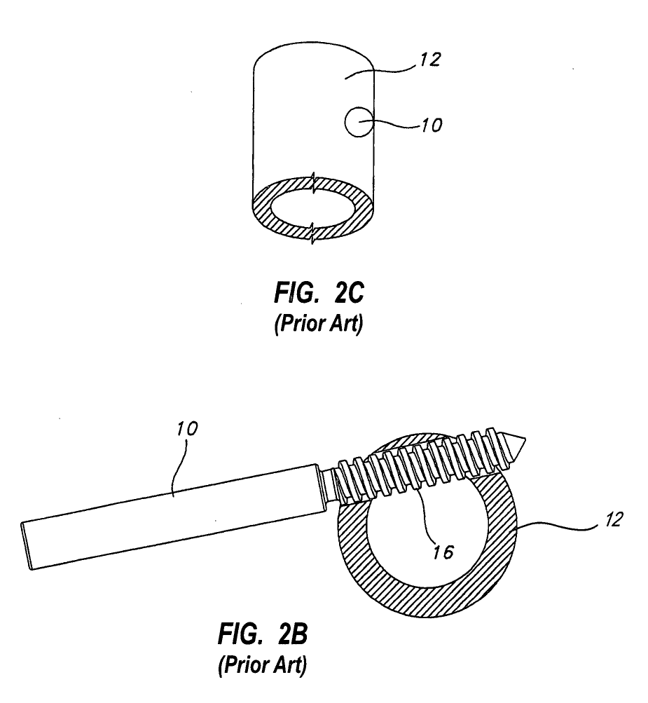

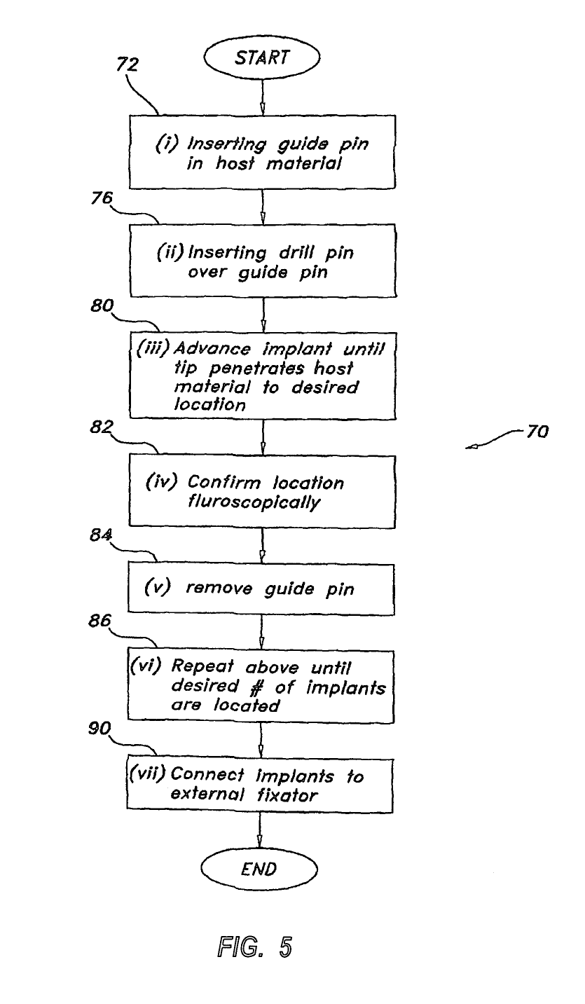

Referring now to FIGS. 3A-3B, a drill-pin implant 30 of the invention has an elongated, cylindrical body 32 extending along a central axis 34. The cylindrical body 32 has opposing end portions 36 and 40; namely, the operative end or tip which penetrates the bone, and a tool engaging end which engages with a hand or power drill, for example. One end portion is an operative end 36 formed so as to be suitable for penetrating bone. The other end portion is a tool engaging end 40 formed so as to be suitable for engagement with, for example, a power or hand drill (not shown). The body 32 has a guide aperture 42 formed along the central axis 34, suitable for engagement over an elongated guide 44 (FIG. 6A) such as a guide pin or guide wire. External threads 46 are provided on the body 32 between the tool engaging end 40 and the operative end 36. The threads 46 are suitable for engaging with the bone 12. The threads 46 are further located adjacent the operative end 36 but may be spaced apart from the operative end 36 a defined distance 50. This spaced-apart distance 50 between the operative end 36 and the external threads 46 generally corresponds to the thickness of the cortex 14 of the type of bone 12 into which bone implant 30 is to be implanted.

Referring now to FIG. 3C, because the bone implant 30 need only remove the material around the guide pin 44, no special form to the operative end 36′ is required: simple, short drill flutes 54 suffice. In the embodiment depicted, each drill flute 54 has a helical curve and is partially bounded by a sharpened, and helically curved cutting edge 58. Preferably, in order to ensure a good cutting efficiency, at least three flutes 54 are provided. Note that in this alternative embodiment, the detail of self-taping flutes 38 on external threads 46 is also shown.

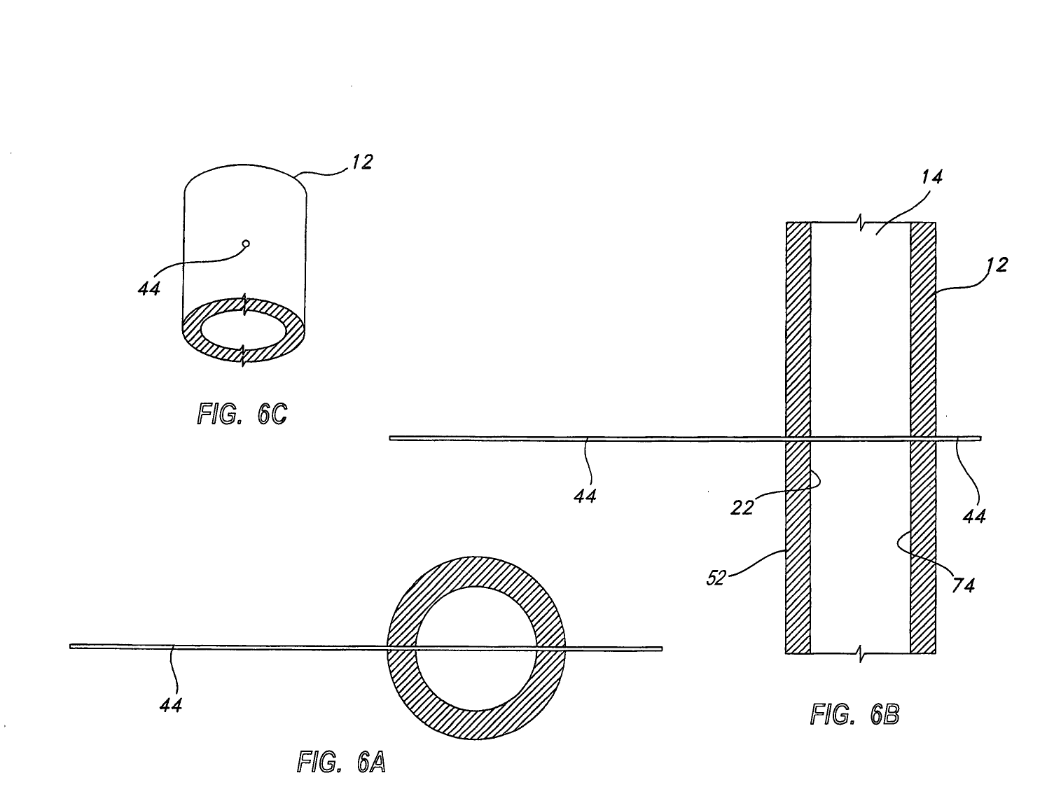

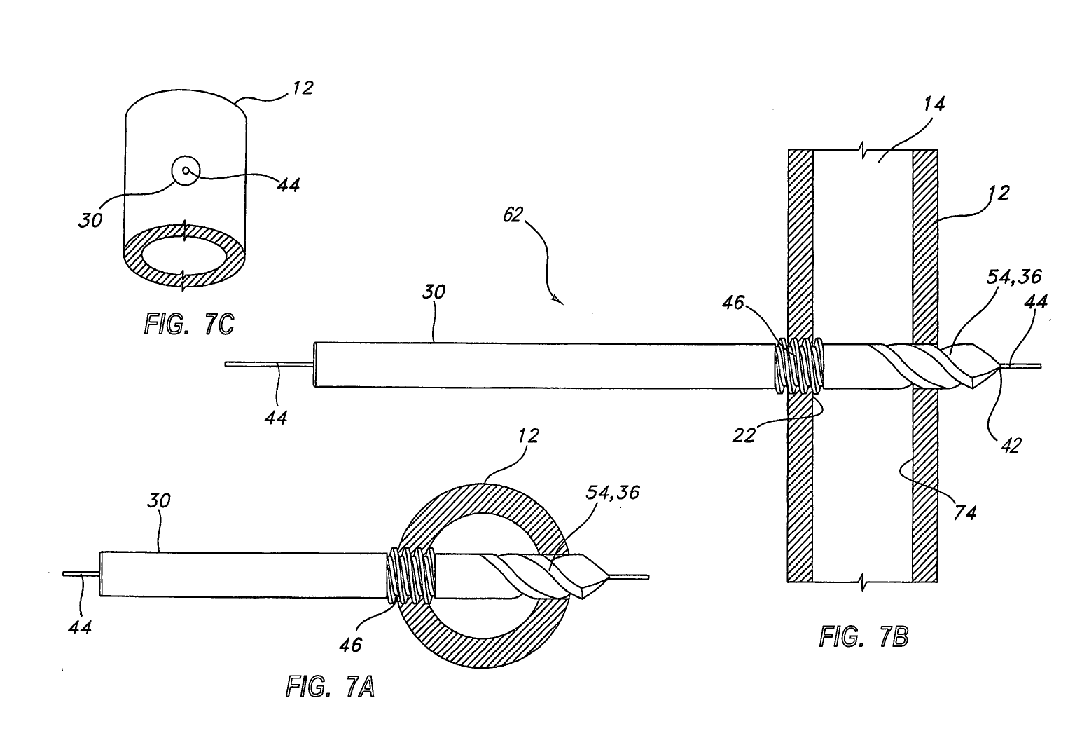

Further, the diameter of the drill-pin implant 30 may be stair-stepped or relieved (not shown) so as to provide clearance after the operative end 36 penetrates a near wall 52 (FIG. 7B) of the bone 12, thus eliminating unnecessary drag, cutting, and thus heat generation.

Drill-pin implant 30 is cannulated to enable precise and concentric implant or pin placement over the guide pin 44 which is then removed. The single use and disposable drill-pin implant 30 features a short cutting flute 54 to avoid damage to soft tissue (e.g., skin 56 and muscle 60 shown in FIG. 2A) during insertion. The external threads 46 are designed to engage the near cortex 52 of the bone 12 and prevent slippage. The threads 46 are optionally coated with hydroxappetite, as such is believed to improve bonding of the host bone 12 to the drill-pin implant 30. The drill-pin implant 30 is provided in a variety of diameters, for example 4, 5, or 6 millimeters; the length is standard because the trailing, tool engaging end 40 of drill-pin implant 30 is cut after it is secured to a frame (not shown). Thus, it is not necessary to stock a wide array of drill-pin implants 30.

One embodiment of a drill-pin system 62 (FIG. 7B) of the invention includes the drill-pin implant 30 and the guide pin 44. The guide pin 44, preferably of a diameter of approximately 1.8 mm (1.2-2 mm is also suitable), is movably disposed within the guide aperture 42 of the drill-pin implant 30. The guide pin 44 is of sufficient length to extend through an incision in the skin 56 and into a bone 12 to a depth that approximates the desired depth of penetration of the drill-pin implant 30.

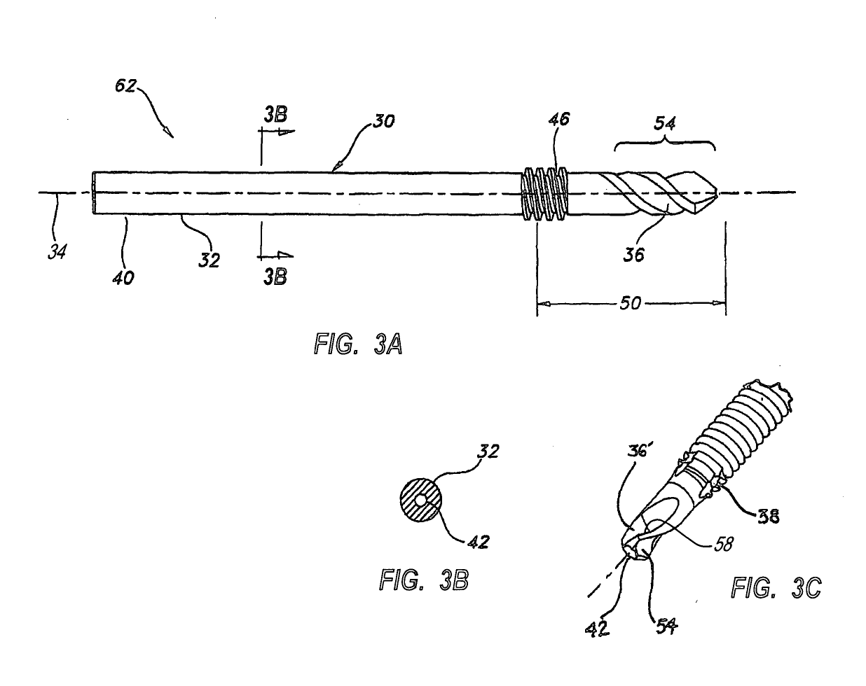

Referring now to FIG. 4, an alternate embodiment of a guide pin 44′ is shown having a fluted end 64 suitable for penetrating bone 12. In alternative embodiments, it is appreciated that any number of different cutting surfaces can be formed at the end of guide pin 44′ so that guide pin 44′ can be directly drilled into bone 12. The guide pin 44′ may also include depth marks 66 along its length suitable to aid a surgeon to determine the depth of penetration of the guide pin 44′ into the patient. The depth marks 66 are preferably laser etched.

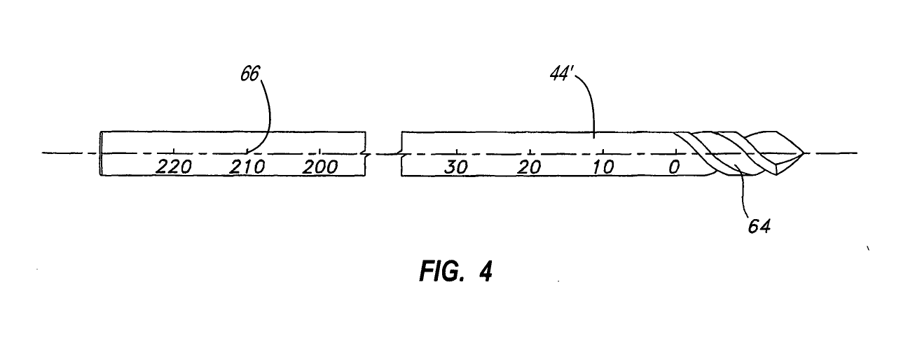

Referring now to FIG. 5, a method of use 70 of drill-pin system 62 is shown. The method 70 includes the following seven steps. Referring now to FIGS. 6A-6C, in a first step 72, the guide pin 44 is inserted, optionally under fluoroscopic guidance, into a host material 12, through a drilled hole (not shown). Symmetrical and central engagement of the near cortex 52 and far cortex 74 ensures subsequent optimal implant placement.

In alternative embodiments where guide pin 44 has a cutting surface formed on the end thereof, such as cutting flutes 64 in FIG. 4, guide pin 44 can be directly drilled into bone 12 to the desired depth. As such a pre-drilled hole is not required. The placement of guide pin 44 within bone 12 requires a much smaller hole than conventional half pins. As such drilling guide pin 44 into bone 12 or pre-drilling bone 12 with a bit can be accomplish with greater precision and less chance that the guide pin 44 or drill bit will walk during drilling.

Referring now to FIGS. 7A-7C, in a second step 76, the drill-pin implant 30 is inserted over the guide pin 44. Drill-pin implant 30 can be positioned over guide pin 44 before, during or after guide pin 44 is positioned within bone 12. Where guide pin 44 is directly drilled into bone 12, drill-pin implant 30 can act to stabilize or reinforce guide pin 44 during the drilling process. Either before or after drill-pin implant 30 is positioned over guide pin 40, bone implant is operably engaged with a turning device (not shown).

With continued reference to FIGS. 7A-7C, in a third step 80, the drill-pin implant 30 is advanced into bone 12 until the tip 36 of drill-pin implant 30 penetrates the bone 12 to its desired location in the host material. Where the host material is bone 12, the drill-pin implant 30 is advanced until the tip 36 penetrates the far cortex 74 and the external threads 46 engage the near cortex 22. Specifically, drill-pin implant 30 is rotated so that the cutting surface, such as cutting edges 58 at operative end 36, cut away bone 12 so as to form a tunnel into or through bone 12. As drill-pin implant 30 continues to bore through bone 12, external threads 46 eventually enter into the bored tunnel. Because external threads 46 have an outer diameter larger than the diameter of the tunnel, external threads 46 engage into the bone surface bounding the tunnel, thereby securing drill-pin implant 30 to bone 12.

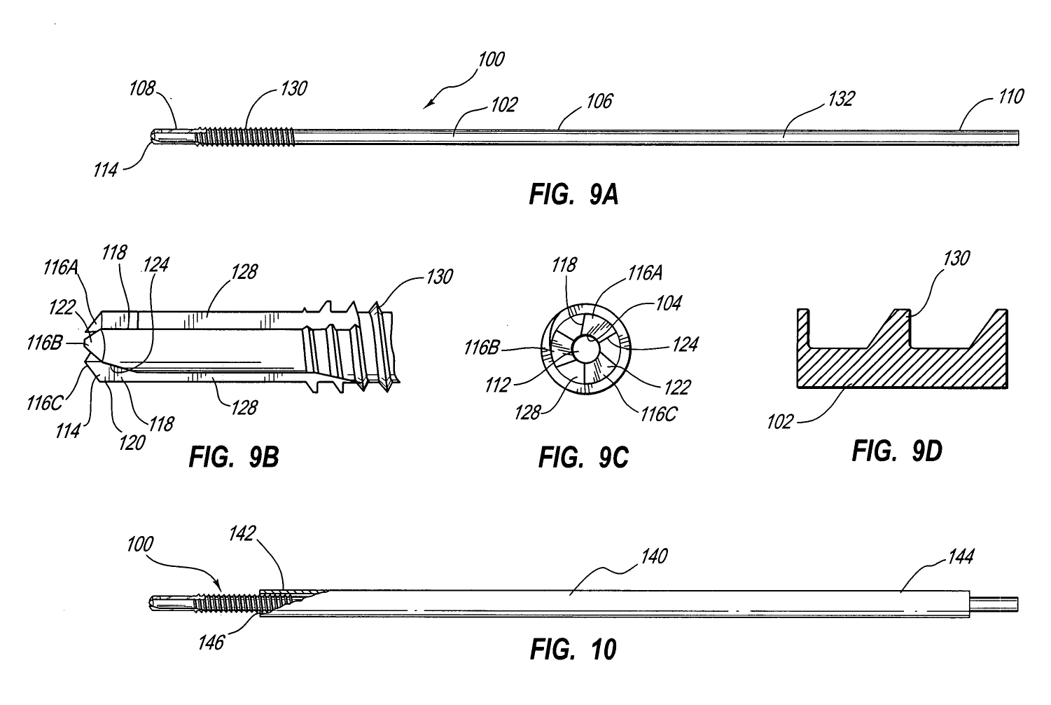

In one alternative method as discussed below with regard to FIG. 10, a drill sleeve 140 can be advanced over drill-pin implant 30 during rotation of drill-pin implant 30 so as to prevent external threads 46 from damaging the surrounding soft tissue. Drill sleeve 140 can thus form a part of drill-pin system 62.

In a fourth step 82, the location of drill-pin implant 30 is optionally confirmed fluoroscopically. Alternatively, the position of guide pin 44 can be confirmed fluoroscopically before insertion of drill-pin implant 30.

In a fifth step 84, the guide pin 44 is removed. In an optional sixth step 86, steps (i) to (vi) are repeated until the desired number of drill-pin implants 30 are located. In this case, each subsequent drill-pin implant 30 (average three per bone segment) is inserted in an identical fashion. In an optional seventh step 90, the drill-pin implant or implants 30 are then connected to an external fixator, and, where necessary, any excess portions of the drill-pin implants are cut off. In a final step, the incision may now be closed.

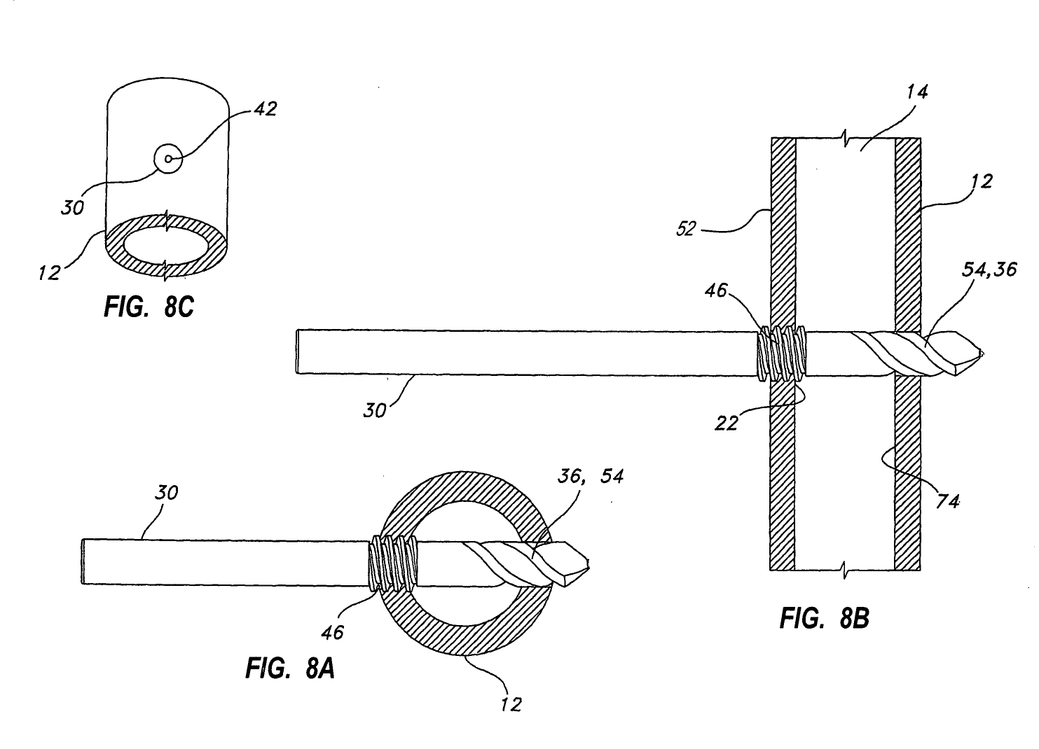

Depicted in FIGS. 9A-9D is an alternative embodiment of a drill-pin implant 100 that can be used in association with guide pin 44. Drill-pin implant 100 comprises a substantially cylindrical body 102 having an interior surface 104 and an exterior surface 106 that each extend between a first end 108 and an opposing second end 110. Interior surface 104 bounds a guide aperture 112 that extends completely through body 102 along a central longitudinal axis thereof. Formed at first end 108 of body 102 is a cutting surface that allows drill-pin implant 100 to bore into bone when drill-pin implant 100 is rotated. Specifically, first end 108 ends at a distal terminus 114. Formed at distal terminus 114 are three radially spaced apart cutting teeth 116A-C. In alternative embodiments, other numbers of teeth 116 can also be used.

Each tooth 116 has a planar front face 118 that terminates at an outside cutting edge 120, a top surface 122 that slopes back toward second end 110, and a back face 124 that is beveled so as to expose the planer front face 1 18 of the next adjacent tooth 116. Formed between each cutting tooth 116 in alignment with each front face 118 is an elongated channel 128 that is recessed into body 102. In the depicted embodiment, each channel 118 is linear and extends longitudinally along body 102. In alternative embodiments, channels 118 can also be curved. In part, channels 118 provide a path through which the bone matter cut by teeth 116 can be removed from the tunnel being bored.

It is appreciated that cutting teeth 116 can have a variety of different configurations and can be replaced with a variety of other types of cutting surfaces that can be used to bore drill-pin implant 100 into bone 12. Cutting teeth 116, however, have been found to be uniquely beneficial when it is desired to mount drill-pin implant 100 at an angle other than normal to the longitudinal axis of the bone. That is, it has been found that unique benefits can be achieved by mounting drill-pin implants 100 into bone so that an inside angle is formed between the longitudinal axis of drill-pin implant 100 and the longitudinal axis of bone 12 that is less then 90° and more commonly between about 15° to about 75° with about 30° to about 60° being more common. Conventional fluted drill bits traditionally have poor initial engagement when drilling at an oblique angle. As such, conventional fluted drill bits can cause relative slow drilling, increased heating of the bone, and potential walking of drill bit. Cutting teeth 116, however, have excellent engagement with a cutting surface when oriented at an oblique angle relative to the surface. As such, use of cutting teeth 116 decreases cutting time while minimizing heat transfer and any potential for walking.

External threads 130 radially outwardly project from exterior surface 106 of body 102 so as to encircle body 102 in a helical pattern. External threads 130 can comprise one or more threads and can have a variety of different pitches. An enlarged view of one embodiment of external threads 130 is shown in FIG. 9D. In the embodiment depicted in FIG. 9B, each channel 128 extends through a lead portion of helical threads 130. Having channel 128 extend through helical threads 130 further assists in removal of cut bone pieces and assists external threads 130 in tapping into bone 12.

If desired, external threads 130 can extend to second end 110. In the depicted embodiment, however, external threads 130 extend along a section of body 102 at a location spaced apart from both opposing ends 108 and 110. A proximal section 132 of body 102 has a smooth, substantially cylindrical configuration extending from external threads 130 to second end 110. The drill-pin implants of the present invention are typically made of metal, such as such as titanium or stainless steel, but other suitable materials can also be used.

As previously mentioned, drill-pin implant 100 can be used in association with guide pin 44 or a variation thereof in the same manner as discussed with regard to drill-pin implant 30. Furthermore, the various drill-pin implants of the present invention can also be used in association with a tubular drill sleeve. For example, depicted in FIG. 10, a tubular drill sleeve 140 is provided having a first end 142 and an opposing second end 144. Drill sleeve 140 bounds a channel 146 that extends entirely through drill sleeve 140 between opposing end 142 and 144. Channel 146 is sized so that drill-pin implant 100 can be slidably received within channel 146 and can selectively pass through channel 146. Drill sleeve 140 has a length that is shorter than the length of drill-pin implant 100 whereas guide pin 44 typically has a length greater than the length of drill-pin implant 100. This enables the rotation tool to engage drill-pin implant 100 when drill sleeve 140 is disposed over drill-pin implant 100. Drill sleeve 140 can be disposable or reusable and can made of metal, plastic, or any other medical grade material.

During use, drill sleeve 140 is initially passed over drill-pin implant 100 so as to cover external threads 130 as drill-pin implant 100 is passed through the soft tissue so as to engage against bone 12. This placement can occur concurrently with or subsequent to positioning guide pin 44 into bone 12. Alternatively, drill sleeve 140 can be positioned over drill-pin implant 100 after drill-pin implant 100 is passed over guide pin 44 but prior to rotation of drill-pin implant 100. Drill sleeve 140 functions as a cover for external threads 130 so that external threads 130 do not directly engage the surrounding soft tissue and potentially tear or damage the soft tissue as drill-pin implant 100 is rotated. After drill-pin implant 100 is securely positioned, sleeve 140 can be removed and discarded or sterilized for reuse.

The inventive drill-pin implants, systems, and methods of the present invention have a number of benefits. For example, the drill-pin implants, guide pins, and/or guide tubes can be designed to be disposable after a single use. As a result, the drill-pin implants are always sharp for drilling, have a known true diameter, and no subsequent sterilization is required.

Furthermore, because the drill-pin implant serves as both the drill and the pin, the cost for purchasing and stocking separate drill bits with their related sharpening and sterilization is eliminated. In addition, carrying an expanded inventory of pins is limited.

By use of the narrow guide pin, the guide pins can be more accurately positioned, thereby avoiding the common problems of walking of the larger drill bits, excessive heating of the bone (e.g., heat necrosis), and eccentric placement of the implants (e.g., high stresses promoting bone fracture).

The inventive system also provides an easier and faster method of implanting the drill-pin implants. Specifically, because the drill bit and pin can be combined in the inventive implants, the inventive method avoids multiple passes of instruments and implants through the skin and muscle. In particular, a separate drilling step may be avoided. In addition, in conventional methods it can be difficult to locate the drilled hole after the drill bit is removed. The present invention has no such problem. The inventive implants can also be used with external fixators (frames) that are commonly employed for the stabilization of long bones during fracture management or deformity correction.

In another advantage, radiographic exposure (e.g., fluoroscopy) is reduced because once the guide pin placement is verified; the only additional documentation required is the depth of drill-pin insertion.

In another advantage, the drill-pin implants may be provided in standard lengths as the excess is easily cut off. Thus, a large variety of half pin lengths and diameters is no longer required.

Multiple variations and modifications are possible in the embodiments of the invention described here. Although certain illustrative embodiments of the invention have been shown and described here, a wide range of modifications, changes, and substitutions is contemplated in the foregoing disclosure. In some instances, some features of the present invention may be employed without a corresponding use of the other features. Accordingly, it is appropriate that the foregoing description be construed broadly and understood as being given by way of illustration and example only, the spirit and scope of the invention being limited only by the appended claims.

1. A dual purpose implant serving as a drill pin and a bone implant, the implant comprising:
an elongated, cannulated body having an interior surface and an exterior surface each extending between a first end and an opposing second end, the interior surface bounding a guide aperture extending between the first end and the opposing second end, the first end of the body being configured to penetrate into bone by cutting away the bone as the body is rotated; and
one or more external threads radially outwardly projecting from the exterior surface of the body, the external threads being configured to engage bone so as to secure the body to the bone.

2. The dual purpose implant of claim 1, wherein one or more cutting flutes are formed at the first end of body, the cutting flutes being configured to cut away bone as the body is rotated.

3. The dual purpose implant of claim 1, wherein one or more cutting teeth are formed at the first end of body, the cutting teeth being configured to cut away bone as the body is rotated.

4. The dual purpose implant of claim 3, further comprising a linear channel recessed into the body and extending from the one or more cutting teeth along a distance of the body.

5. The dual purpose implant of claim 1, wherein the external threads are disposed between the first end and the second end of the body.

6. The dual purpose implant of claim 1, wherein the one or more external threads circle the body in a helical configuration.

7. The dual purpose implant of claim 6, further comprising a cutting tooth formed at the first end of the body and a linear channel extending from the cutting tooth through a portion of the external threads having the helical configuration.

8. The dual purpose implant of claim 6, wherein the one or more external threads are self-tapping threads

9. The dual purpose implant of claim 1, wherein the second end of the body has a smooth, cylindrical configuration.

10. A drill-pin system comprising:
an implant as recited in claim 1;
a guide removably disposed within the guide aperture of the body.

11. The drill pin system of claim 10, wherein the guide comprises an elongated guide wire or a guide pin.

12. The drill pin system of claim 10, wherein the guide has a leading end with a cutting edge formed thereat so that the guide can be directly drilled into bone.

13. The drill pin system of claim 10, wherein the guide has depth marks along its length suitable to aid a surgeon to determine the depth of penetration of the guide into the patient.

14. The drill system of claim 10, further comprising a tubular drill sleeve, the implant being removably disposed within the drill sleeve.

15. A drill pin system comprising:
a drill-pin implant having an elongated, cannulated body with an interior surface and an exterior surface each extending between a first end and an opposing second end, the interior surface bounding a guide aperture extending between the first end and the opposing second end, the first end of the body having a cutting edge formed thereat, one or more external threads radially outwardly projecting from the exterior surface of the body; and
an elongated guide removably disposed within the guide aperture of the drill-pin implant.

16. The drill pin system of claim 15, wherein the guide has a leading end with a cutting edge formed thereat so that the guide can be directly drilled into bone.

17. The drill pin system of claim 15, further comprising a tubular drill sleeve, the drill-pin implant being removably disposed within the drill sleeve.

18. A method for mounting a drill-pin implant on a bone, the method comprising the steps of:
positioning the end of a guide within a bone;
rotating a cannulated drill-pin implant encircling at least a portion of the guide so that a lead end of the drill-pin implant bores into the bone;
advancing the drill-pin implant further into the bone so that external threads of the drill-pin implant engage the bone, thereby securing the drill-pin implant to the bone; and
removing the guide from within the drill-pin implant.

19. The method of claim 18, wherein the guide comprises an elongated guide pin or guide wire.

20. The method of claim 18, wherein the step of positioning the end of the guide within the bone comprises directly drilling the guide into the bone.

21. The method of claim 18, wherein at least a portion the guide is disposed within the drill-pin implant while the end of the guide is being positioned within the bone.

22. The method of claim 18, wherein the guide is disposed within the drill-pin implant and the drill-pin implant is disposed within a tubular sleeve while the end of the drill-pin implant is being positioned into the bone.

23. The method of claim 18, wherein the lead end of the drill-pin implant bores into the bone by cutting away the bone so as to form a tunnel into the bone.

24. The method of claim 23, wherein the external threads engage the wall bounding the tunnel.

25. The method of claim 18, further comprising securing the drill-pin implant to an external frame structure.

26. The method of claim 18, further comprising cutting off a second end of the drill-pin implant.
